(19) United States
(12) Patent Application Publication
(10) Pub. No.: US 2022/0386660 A1
(43) Pub. Date: Dec. 8, 2022

(54) METHOD FOR OBTAINING AN OAT-BASED PRODUCT

(71) Applicant: Novozymes A/S, Bagsvaerd (DK)

(72) Inventors: Benjamin Thieringer, Arlesheim (CH); Alessandro Palumbo, Basel (CH)

(73) Assignee: Novozymes A/S, Bagsvaerd (DK)

(21) Appl. No.: 17/773,703

(22) PCT Filed: Nov. 19, 2020
(86) PCT No.: PCT/EP2020/082673

§ 371 Date: May 2, 2022

(30) Foreign Application Priority Data
Nov. 22, 2019 (EP) 19210996.5

(51) Int. Cl. A23L 7/104

(52) U.S. Cl. CPC A23L 7/107; A23V 2002/00

(57) ABSTRACT

The present invention relates to use of enzymes having alpha-amylase activity for obtaining a hydrolysed oat material.

### REFERENCE TO SEQUENCE LISTING

This application contains a Sequence Listing in computer readable form. The computer readable form is incorporated herein by reference.

### FIELD OF THE INVENTION

The present invention relates to use of enzymes having alpha-amylase activity for obtaining a hydrolysed oat material.

### BACKGROUND OF THE INVENTION

There is a growing interest in food products made from oats. Oats are perceived as healthy for a number of reasons: They are a great source of important vitamins, minerals, fiber (β-glucans), antioxidants as well as essential amino acids. Health benefits which have been associated with intake of oats include weight loss, lower blood cholesterol levels and a reduced risk of heart disease.

Oat-based food products or oat-based ingredients to be included in food products include oat-based beverages, oat-based syrups/concentrates/extracts, e.g., having at least 20% dry solids, fermented oat-based products and oat-based ice-creams.

U.S. Pat. No. 4,282,319 discloses enzymatic modification of whole grain with a protease and an amylase.

U.S. Pat. No. 4,996,063 discloses enzymatic modification of ground oat products with an alpha-amylase.

U.S. Pat. No. 5,686,123 discloses enzymatic modification of a cereal suspension by sequential use of a beta-amylase, which has no glucanase and proteinase activity, and an alpha-amylase, which also has no glucanase and proteinase activity.

W000/22938 and WO02/065855 both disclose enzymatic modification of a cereal suspension using at least one hydrolase having the ability to hydrolyze alpha-glycosidic bonds and having no glucanase and proteinase effect. The hydrolase may be selected from the group consisting of beta-amylase, alpha-amylase, amyloglucosidase and pullulanase, with the proviso that when the enzyme preparation comprises beta-amylase or alpha-amylase there is a mixture of at least one other of the named alpha-glycosidic hydrolases.

WO2011/070057, WO2011/070083 and WO2011/070086 disclose enzymatic modification of a whole grain component with an alpha-amylase which shows no hydrolytic activity towards dietary fibers, and optionally an amyloglucosidase which also shows no hydrolytic activity towards dietary fibers.

WO2010/036515 discloses processes using blends of alpha-amylases for starch liquefaction and saccharification. Use of an enzyme preparation having beta-glucanase activity is not disclosed.

In general, to convert the oat kernels to an oat-based food product, an oat-based beverage or an oat-based ingredient to be included in a food product, the starch in the oat kernel must be hydrolysed. The conversion of the oat starch may include a gelatinisation step which involves the dissolution of the nanogram-sized starch granules to form a viscous suspension, a liquefaction step which involves the partial hydrolysis of the starch with concomitant loss in viscosity, and possibly a saccharification step which involves the production of glucose and maltose by further hydrolysis.

Gelatinization is normally attained by heating, whereas liquefaction and possible saccharification often involves the use of enzymes. Since high temperature is preferably used for the gelatinization, it is an advantage if the liquefaction can be performed also at high temperature. In that case, gelatinization and liquefaction can be performed as one step.

The standard production process for oat-based products used industrially today uses a bacterial endo-alpha-amylase for liquefaction. In many cases though, the oat kernel is not fully hydro- lysed thus leading to a waste of raw material.

It is an object of the present invention to identify improved processes for production of a hydrolysed oat-based product which increases the yield, e.g., by optimizing the viscosity to obtain a better separation of the liquid and solid phase using, e.g., a decanter or a centrifuge, while at the same time helping manufacturers to achieve the desired viscosity/mouthfeel in the final product.

In the industry today, gelatinization and liquefaction are preferably carried out at high temperature to fully gelatinize the oat starch (amylose and amylopectin). A fully gelatinized oat starch leads to a higher yield since the substrate is accessible for the added amylases.

The standard industrial production process for oat-based products uses glucoamylase (also known as amyloglucosidase or AMG) for saccharification, see e.g. Lebensmittel Technik November 2018, pp. 10-13. Use of glucoamylase for saccharification leads to a relatively sweet product rich in glucose. To reduce the perceived sweetness and amount of glucose, the glucoamylase is sometimes replaced with another saccharifying enzyme such as Fungamyl (fungal alpha-amylase from Aspergillus oryzae).

Enzymes for liquefaction and saccharification are usually applied at two different temperatures, e.g., liquefaction at about 70-100° C. and saccharification at about 40-65° C. Such temperature adjustment is costly due to energy consumption, time, equipment and complexity of the process.

It is a further object of the present invention to identify improved processes for production of a hydrolysed oat-based product which is not too sweet.

### SUMMARY OF THE INVENTION

The present inventors have found that by combining in an oat liquefaction step at least one heat tolerant bacterial endo-alpha-amylase, e.g., obtained from Bacillus licheniformis or Bacillus stearothermophilus, and at least one enzyme preparation having beta-glucanase activity, e.g., a preparation of an endo-alpha-amylase obtained from Bacillus amyloliquefaciens having beta- glucanase side activity or a cellulolytic enzyme preparation obtained from Trichoderma reesei, an increased yield and/or improved viscosity can be obtained.

If using in the oat liquefaction process only a bacterial endo-alpha-amylase preparation from Bacillus amyloliquefaciens, a product with a very low viscosity and watery mouthfeel is produced. If using only a heat tolerant bacterial endo alpha-amylase either from Bacillus licheniformis or Bacillus stearothermophilus, a product with a high viscosity and sandy mouthfeel is produced. The combination of the two enzymes will enable producers of oat-based products to achieve the desired viscosity/mouthfeel.

The invention therefore provides a method for obtaining a hydrolysed oat material which comprises:

(a) obtaining a slurry of an oat material in water, where the ratio of oat material to water is 1:3 to 1:8 (w/w), and

(b) liquefying the slurry of step (a) at a temperature of 70-90° C. with at least one heat tolerant bacterial endo-alpha-amylase and at least one enzyme preparation having beta-glucanase activity.

The inventors have further found that by combining a liquefying bacterial endo-alpha-amylase, e.g., an endo-alpha-amylase obtained from Bacillus amyloliquefaciens, and a saccharifying bacterial maltogenic alpha-amylase, e.g., a bacterial maltogenic alpha-amylase obtained from Bacillus stearothermophilus, liquefaction and saccharification can be performed as one step at a temperature of 70-90° C., and the resulting product has a moderate perceived sweetness and an increased amount of maltose relative to glucose.

The invention therefore further provides a method for obtaining a hydrolysed oat material which comprises

(a) obtaining a slurry of a heat-treated oat material in water, and

(b) liquefying and saccharifying the slurry of step (a) in one step at a temperature of 70-90° C. with at least one bacterial endo-alpha-amylase and at least one bacterial maltogenic alpha-amylase.

### DETAILED DESCRIPTION OF THE INVENTION

First aspect

In a first aspect, the present invention provides a method for obtaining a hydrolysed oat material which comprises:

(a) obtaining a slurry of an oat material in water, where the ratio of oat material to water is 1:3 to 1:8 (w/w), and

(b) liquefying the slurry of step (a) at a temperature of 70-90° C. with at least one heat tolerant bacterial endo-alpha-amylase and at least one enzyme preparation having beta-glucanase activity.

The oat material may be heat treated.

The oat material may be oat flour such as heat-treated oat flour or it may be milled oat kernels such as de-hulled and heat-treated oat kernels which have been wet-milled or it may be any other oat material known in the art.

In a preferred embodiment, the oat material is oat flour, preferably heat-treated oat flour.

In step (a) the ratio of oat material to water is preferably 1:4 to 1:6.

Step (b) may be performed for 5-60 minutes, preferably 15-45 minutes.

The heat tolerant bacterial alpha-amylase is preferably obtained from, or is a variant of a heat tolerant endo-alpha-amylase obtained from, Bacillus, preferably from Bacillus licheniformis or Bacillus stearothermophilus.

Examples of heat tolerant bacterial alpha-amylases are Termamyl® Classic or Termamyl® SC available from Novozymes A/S.

“Heat tolerant” in the context of the present invention means that the enzyme can resist irreversible thermal inactivation.

The heat tolerant bacterial endo-alpha-amylase may retain at least 50% of its activity after incubation in 20% oat flour at 85° C. for 30 minutes, preferably at 90° C. for 30 minutes.

A particularly preferred heat tolerant bacterial endo-alpha-amylase is the endo-alpha-amylase of SEQ ID NO: 1. Another preferred heat tolerant bacterial endo-alpha-amylase is the endo-alpha-amylase of SEQ ID NO: 2.

In a preferred embodiment, the heat tolerant bacterial endo-alpha-amylase has at least 70% sequence identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% sequence identity to SEQ ID NO: 1.

In another preferred embodiment, the heat tolerant bacterial endo-alpha-amylase has at least 70% sequence identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% sequence identity to SEQ ID NO: 2.

The term “identity” is the relatedness between two amino acid sequences or between two nucleotide sequences. For purposes of the present invention, the degree of identity between two amino acid sequences is determined using the Needleman-Wunsch algorithm (Needleman and Wunsch, 1970, J. Mol. Biol. 48: 443-453) as implemented in the Needle program of the EM-BOSS package (EMBOSS: The European Molecular Biology Open Software Suite, Rice et al., 2000, Trends in Genetics 16: 276-277), preferably version 3.0.0 or later. The optional parameters used are gap open penalty of 10, gap extension penalty of 0.5, and the EBLOSUM62 (EM-BOSS version of BLOSUM62) substitution matrix. The output of Needle labelled “longest identity” (obtained using the —nobrief option) is used as the percent identity and is calculated as follows:

(Identical Residues×100)/(Length of Alignment—Total Number of Gaps in Alignment)

The heat tolerant bacterial endo-alpha-amylase may be added in the range of 10-10,000 KNU, preferably 50-2,000 KNU, even more preferably 200-250 KNU per kg oat flour.

One Kilo Novo alpha amylase Unit (KNU) equals 1000 NU. One KNU is defined as the amount of enzyme which, under standard conditions, dextrinizes 5.26 g starch dry substance Merck Amylum solubile.

The enzyme preparation having beta-glucanase activity may be, e.g., a preparation of an endo-alpha-amylase obtained from Bacillus, preferably from Bacillus amyloliquefaciens, having beta-glucanase side activity or a cellulolytic enzyme preparation obtained from Trichoderma reesei.

Examples of enzyme preparations having beta-glucanase activity are BAN or Celluclast® available from Novozymes A/S.

In a preferred embodiment, the enzyme preparation having beta-glucanase activity is a preparation of an endo-alpha-amylase obtained from Bacillus, preferably from Bacillus amyloliquefaciens, having beta-glucanase side activity.

Such endo-alpha-amylase may have at least 70% sequence identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% sequence identity to SEQ ID NO: 3.

A preparation of such endo-alpha-amylase may comprise 5-10 FBG/KNU beta-glucanase activity.

One Fungal Beta-Glucanase unit (FBG) is the amount of enzyme that produces reducing carbohydrate equivalent to 1 μmol glucose per minute under the standard conditions as follows: temperature 50° C., pH 5, 5 g/L beta-glucan as substrate, reaction time 1200 s.

A preparation of such endo-alpha-amylase may comprise 1-3 BGU/KNU beta-glucanase activity.

One Beta-Glucanase Unit (BGU) is the amount of enzyme that produces reducing carbohydrate equivalent to 1 μmol glucose per minute under the conditions by the reduced sugar Somoguy Nelson method.

In another preferred embodiment, the enzyme preparation having beta-glucanase activity is a cellulolytic enzyme preparation obtained from Trichoderma reesei.

The enzyme preparation having beta-glucanase activity may be added in the range of 1-1,000 BGU, preferably 2-200 BGU per kg oat flour.

The enzyme preparation having beta-glucanase activity may be added in the range of 1-5,000 FBG, preferably 3-1,000 FBG per kg oat flour.

After step (b), a saccharification step is preferably performed by incubating with a glucoamylase at 40-65° C., preferably at 55-60° C., for 5-60 minutes, preferably for 10-30 minutes.

The glucoamylase may be added at a concentration of 50-1000 AGU/kg oat material.

One Glucoamylase Unit (AGU) is defined as the amount of enzyme, which hydrolyzes 1 micromole maltose per minute under the standard conditions 37° C., pH 4.3, substrate: maltose 23.2 mM, buffer: acetate 0.1 M, reaction time 5 minutes.

After the treatment with the glucoamylase, the enzymes may be inactivated by heat treatment. E.g., by increasing the temperature to 95° C. for 10 minutes. After inactivation, the hydrolysates may be cooled.

The liquid and the solid phase may be separated, e.g., by centrifugation.

The liquid phase may be formulated using for instance sodium chloride (NaCl), oil and flavouring agents. It may be homogenized. It may be UHT or ESL treated and aseptically packed.

The final product may be sold as an oat-based beverage. Alternatively, it may be further processed into a food product, such as a fermented oat-based product or an oat-based ice cream, or it may be used as an ingredient in a food product.

Second aspect

In a second aspect, the present invention provides a method for obtaining a hydrolysed oat material which comprises:

(a) obtaining a slurry of an oat material in water, and

(b) liquefying and saccharifying the slurry of step (a) in one step at a temperature of 70-90° C. with at least one bacterial endo-alpha-amylase and at least one bacterial maltogenic alpha-amylase.

The oat material may be heat treated.

The oat material may be oat flour such as heat-treated oat flour or it may be milled oat kernels such as de-hulled and heat-treated oat kernels which have been wet-milled or it may be any other oat material known in the art.

In a preferred embodiment, the oat material is oat flour, preferably heat-treated oat flour.

In step (a) the ratio of oat material to water may be 1:3 to 1:8 (w/w), preferably 1:4 to 1:6.

Step (b) may be performed for 5-60 minutes, preferably 15-45 minutes.

The bacterial endo-alpha-amylase is preferably obtained from, or is a variant of an endo-alpha-amylase obtained from, Bacillus, preferably from Bacillus amyloliquefaciens.

An example of a bacterial endo-alpha-amylase is BAN available from Novozymes A/S.

A particularly preferred bacterial endo-alpha-amylase is the endo-alpha-amylase of SEQ ID NO: 3.

In a preferred embodiment, the bacterial endo-alpha-amylase has at least 70% sequence identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% sequence identity to SEQ ID NO: 3.

The bacterial endo-alpha-amylase may be added in the range of 50-50,000 KNU, preferably 100-10,000 KNU, even more preferably 500-2,000 KNU per kg oat flour.

A “maltogenic alpha amylase” is understood as an enzyme classified in EC 3.2.1.133. The enzymatic activity does not require a non-reducing end on the substrate and the primary enzymatic activity results in the degradation of amylopectin and amylose to maltose and longer malto-dextrins. It is able to hydrolyze amylose and amylopectin to maltose in the alpha-configuration.

The bacterial maltogenic alpha-amylase is preferably obtained from, or is a variant of a malto-genic alpha-amylase obtained from, Bacillus, preferably from Bacillus stearothermophilus.

A particularly preferred bacterial maltogenic alpha-amylase is Maltogenase® available from No- vozymes A/S.

The bacterial maltogenic alpha-amylase may be heat tolerant. It may retain at least 50% of its activity after incubation in 20% oat flour at 80° C. for 30 minutes.

A particularly preferred bacterial maltogenic alpha-amylase is the maltogenic alpha-amylase of SEQ ID NO: 4.

In a preferred embodiment, the bacterial maltogenic alpha-amylase has at least 70% sequence identity, such as at least 75%, such as at least 80%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or even 100% sequence identity to SEQ ID NO: 4.

The bacterial maltogenic alpha-amylase may be added in the range of 500-500,000 MANU, preferably 1,000-100,000 MANU, even more preferably 5,000-50,000 MANU per kg oat flour.

One Maltogenic Amylase Novo Unit (MANU) is the amount of enzyme which under standard conditions cleaves one μmol maltotriose per minute. The standard conditions are 10 mg/ml maltotriose, 37° C., pH 5.0, 30 minutes reaction time.

After step (b), the enzymes may be inactivated by heat treatment. E.g., by increasing the temperature to 95° C. for 10 minutes. After inactivation, the hydrolysates may be cooled.

The hydrolysed oat material obtained may comprise maltose:glucose in a ratio of at least 1, preferably at least 2, more preferably at least 4 (w/w).

The desired maltose production and the desired relative sweetness will depend on, e.g., the specific product, the region where it is to be sold as well as consumer preferences.

The liquid and the solid phase may be separated, e.g., by centrifugation.

The liquid phase may be formulated using for instance sodium chloride (NaCl), oil and flavouring agents. It may be homogenized. It may be UHT or ESL treated and aseptically packed.

The final product may be sold as an oat-based beverage. Alternatively, it may be further processed into a food product, such as a fermented oat-based product or an oat-based ice cream, or it may be used as an ingredient in a food product.

### EXAMPLES

### Example 1: Treatment of oat flour with heat tolerant endo-alpha-amylase from Bacillus licheniformis combined with endo-alpha-amylase from Bacillus amyloliquefaciens

A heat tolerant endo-alpha-amylase from Bacillus licheniformis (SEQ ID NO: 1) which does not have beta-glucanase side activity (termed BLA) and an endo-alpha-amylase preparation from Bacillus amyloliquefaciens (SEQ ID NO: 3) which has beta-glucanase side activity (termed BAA) were added to water in the amounts shown in Table 1 below. Heat-treated oat flour was mixed with the water comprising enzyme in a ratio of 50 g oat flour to 250 g of water.

In a next step the mixture of water, enzyme and oat was heated to a temperature of 85° C. for 30 minutes (liquefaction). Afterwards the hydrolysate was cooled down to 60° C. and AMG was added at a concentration of 300 AGU/kg oat flour for saccharification. The hydrolysate was held for 15 minutes at 60° C. followed by inactivation of the enzymes by increasing the temperature to 95° C. for 10 minutes. After inactivation, the hydrolysates were cooled to <60° C. for centrifugation. Separation of the liquid and solid phase was made by means of a centrifuge using 3950 RPM for 5 minutes. The amount of supernatant was determined, and the results shown in Table 1 below.

TABLE 1

| BLA [KNU]/BAA [KNU]* | 240/0 | 238/10 | 235/19 | 233/29 | 230/38 | 228/48 | 216/96 | 0/960 |
|---|---|---|---|---|---|---|---|---|
| Supernatent [ml] | 223 | 244.8 | 245.8 | 245.8 | 244.9 | 245.0 | 246.5 | 238.0 |
| °Brix | 12.6 | 12.8 | 12.7 | 12.7 | 12.7 | 12.8 | 12.8 | 12.2 |
| Total solid in supernatant [g] | 28.1 | 31.2 | 31.3 | 31.2 | 31.1 | 31.3 | 31.6 | 29.1 |
| Total solid in supernatant [%] | 100% | 111% | 111% | 111% | 111% | 112% | 113% | 104% |
| Viscosity at 20° C. [mPa*s] | >120 | 6.4 | 4.4 | 3.7 | 3.4 | 3.2 | 2.8 | 2.2 |

*The KNU is per kg oat flour. BAA comprises 8.6 FBG/KNU and 1.8 BGU/KNU

As shown in Table 1, the combination of BLA and BAA increases the total solid content of the supernatant after centrifugation and their combined performance is superior to the one showed when the two enzymes are added individually. Further, when BLA is used alone, the viscosity is high which may give a sandy mouthfeel. When BAA is used alone, the viscosity is low which may give a watery mouthfeel. Combination of the two enzymes makes it possible to obtain a viscosity which is not too low and not too high.

Optionally the product could be formulated using for instance sodium chloride (NaCl), oil and flavoring, homogenized, UHT or ESL treated and aseptically packed.

### Example 2: Treatment of oat flour with heat tolerant endo-alpha-amylase from Bacillus licheniformis combined with enzyme having beta-glucanase activity

A heat tolerant endo-alpha-amylase from Bacillus licheniformis (SEQ ID NO: 1) which does not have beta-glucanase side activity (termed BLA) and an endo-alpha-amylase preparation from Bacillus amyloliquefaciens (SEQ ID NO: 3) which has beta-glucanase side activity (termed BAA) were added to water in the amounts shown in Table 2 below. Heat-treated oat flour was mixed with the water comprising enzyme in a ratio of 50 g oat flour to 250 g of water.

In a next step the mixture of water, enzyme and oat was heated to a temperature of 85° C. for 30 minutes (liquefaction). Afterwards the hydrolysate was cooled down to 60° C. and AMG was added at a concentration of 300 AGU/kg oat flour for saccharification. The hydrolysate was held for 15 minutes at 60° C. followed by inactivation of the enzymes by increasing the temperature to 95° C. for 10 minutes. After inactivation, the hydrolysates were cooled to <60° C. for centrifugation. Separation of the liquid and solid phase was made by means of a centrifuge using 3950 RPM for 5 minutes. The amount of supernatant was determined, and the results shown in Table 2 below.

In a further experiment, BLA was combined with Celluclast® (cellulolytic enzyme preparation obtained from Trichoderma reesei having a beta-glucanase activity) in the dosages shown in Table 2 to show that the effect of combining BLA with BAA is due to the beta-glucanase side activity of BAA.

The experiment was done in the same way as the experiment right above and the results are shown in Table 2 below.

Table 2 also shows data on use of heat tolerant endo-alpha-amylase from Bacillus stearothermophilus (SEQ ID NO: 2) which does not have beta-glucanase side activity (termed BSA) with-out BAA or Celluclast.

TABLE 2
All enzyme activity units are per kg oat flour

| | | | | | | Total solid in | Total solid in | Viscosity |
|---|---|---|---|---|---|---|---|---|
| BLA | BSA | BAA* | Celluclast | Supernatant | | supernatant | supernatant | at 20° C. |
| [KNU] | [KNU] | [KNU] | [BGU/FBG] | [mL] | °Brix | [g] | [%] | [mPa*s] |
| 240 | — | — | — | 225.5 | 12.2 | 27.6 | 100% | >120 |
| 239 | — | 5 | — | 244.4 | 12.5 | 30.6 | 111% | 4.2 |
| 237 | — | 12 | — | 245.7 | 12.7 | 31.1 | 113% | 2.9 |
| 234 | — | 24 | — | 246 | 12.7 | 31.3 | 113% | 2.5 |
| — | — | 960 | — | 229.6 | 12.3 | 28.1 | 102% | 2.1 |
| 239 | — | — | 2.2/3.7 | 243.9 | 12.5 | 30.5 | 111% | 6.2 |
| 237 | — | — | 5.5/9.3 | 247.2 | 12.5 | 31 | 112% | 4.1 |
| 234 | — | — | 11/18.5 | 248.7 | 12.6 | 31.4 | 114% | 3.3 |
| 231 | — | — | 16.5/27.8 | 248.5 | 12.7 | 31.5 | 114% | 2.9 |
| 228 | — | — | 22/37 | 248.7 | 12.7 | 31.6 | 115% | 2.6 |
| — | 1000 | — | — | 224.8 | 12.7 | 28.6 | 100% | >120 |
| — | 1500 | — | — | 225.3 | 12.7 | 28.7 | 101% | >120 |
| — | 2000 | — | — | 226.0 | 12.8 | 28.9 | 101% | >120 |

*BAA comprises 8.6 FBG/KNU and 1.8 BGU/KNU

As shown in Table 2, Celluclast® combined with BLA gives the same yield increase and improved viscosity as BAA combined with BLA. BSA used alone gives comparable yield and viscosity as BLA used alone.

Optionally the liquid phase product could be formulated using for instance sodium chloride (NaCl), oil and flavoring, homogenized, UHT or ESL treated and aseptically packed.

### Example 3: Treatment of oat flour with endo-alpha-amylase from Bacillus amyloliquefaciens and maltogenic alpha-amylase

50 g of heat-treated oat flour was mixed with 250 g of water (total weight 300 g).

Endo-alpha-amylase from Bacillus amyloliquefaciens (SEQ ID NO: 3) termed BAA and malto-genic alpha-amylase from Bacillus stearothermophilus (SEQ ID NO: 4) termed MAA were added to it in the amounts shown in Table 3 below. The mixture was heated to 80° C. for 30 minutes (liquefaction and saccharification).

In a parallel experiment, BAA was added first and the mixture incubated at 80° C. for 30 minutes (liquefaction). Afterwards the hydrolysate was cooled down to 60° C. and AMG was added at a concentration of 300 AGU/kg oat flour for saccharification. The hydrolysate was held for 15 minutes at 60° C.

In another parallel experiment, BAA was added first and the mixture incubated at 80° C. for 30 minutes (liquefaction). Afterwards the hydrolysate was cooled down to 55° C. and Fungamyl was added at a concentration of 2400 FAU-F/kg oat flour for saccharification. The hydrolysate was held for 15 minutes at 55° C.

Temperature of the mixture was then increased to 95° C. for 15 minutes to inactivate the enzymes. Water was added to reach 300 g (original total weight) to compensate the water evaporation.

The solid and liquid phase of the mixture were separated by means of a centrifuge running at 3000 RPM for 15 minutes.

The amount of maltose and glucose present in the liquid phase was measured using Thermo Fisher's High Pressure Anion Exchange Pulsed Amperometric Detection method.

Relative sweetness was calculated using sweetness coefficients indicated in the “Lehrbuch der Lebensmittelchemie—Springer—Belitz—Grosch—Schieberle” (table 4.10; page 246).

The results are shown in Table 3.

TABLE 3
All enzyme activity units are per kg oat flour

| AMG | | Fungamyl | | | | Total | |
|---|---|---|---|---|---|---|---|
| 300 L | BAA | 800 L | MAA | Glucose | Maltose | sugars | Relative |
| [AGU] | [KNU] | [FAU-F] | [MANU] | [g/100 g] | [g/100 g] | [g/100 g] | sweetness |
| 300 | 960 | — | — | 2.9 | 1.1 | 4.0 | 2.5 |
| — | 960 | 2400 | — | 0.4 | 4.3 | 4.7 | 2.3 |
| — | 960 | — | 10000 | 0.4 | 2.2 | 2.6 | 1.3 |
| — | 960 | — | 20000 | 0.5 | 3 | 3.5 | 1.7 |
| — | 960 | — | 30000 | 0.6 | 3.7 | 4.3 | 2.1 |

As can be seen, the combination of a bacterial endo-alpha-amylase with a bacterial maltogenic alpha-amylase allows for performing liquefaction and saccharification in one step.

Table 3 further shows that Fungamyl and maltogenic alpha-amylase are producing more maltose and less glucose compared to AMG. Maltose has a lower relative sweetness compared to glucose. According to “Lehrbuch der Lebensmittelchemie — Springer — Belitz — Grosch — Schieberle” (table 4.10, page 246), maltose has a relative sweetness of 0.46 and Glucose 0.69.

Therefore, a less sweet oat drink can be produced by keeping the total amount of sugars constant by using maltogenic alpha-amylase.

Example on how to calculate relative sweetness: 960 KNU of BBA combined with 30,000 MANU of MAA results in 0.6 g glucose/100 g*0.69+3.7 g maltose/100 g*0.46=2.1.

1. A method for obtaining a hydrolysed oat material which comprises
(a) obtaining a slurry of an oat material in water, where the ratio of oat material to water is 1:3 to 1:8 (w/w), and
(b) liquefying the slurry of step (a) at a temperature of 70-90° C. with at least one heat tolerant bacterial endo-alpha-amylase and at least one enzyme preparation having beta-glucanase activity.

2. The method of claim 1, wherein the enzyme preparation having beta-glucanase activity is a preparation of an endo-alpha-amylase obtained from Bacillus having beta-glucanase side activity.

3. The method of claim 1, wherein the enzyme preparation having beta-glucanase activity is a cellulolytic enzyme preparation obtained from Trichoderma reesei.

4. The method of claim 1, wherein the enzyme preparation having beta-glucanase activity is added at a dosage of 1-1,000 BGU per kg oat material.

5. The method of claim 1, wherein the heat tolerant bacterial endo-alpha-amylase is obtained from, or is a variant of a heat tolerant endo-alpha-amylase obtained from, Bacillus.

6. The method of claim 1, wherein the heat tolerant bacterial endo-alpha-amylase has at least 70% sequence identity to SEQ ID NO: 1 or 2.

7. The method of claim 1, wherein step (b) is performed for 5-60 minutes.

8. The method of claim 1, wherein after step (b) a saccharification step is performed by incubating with a glucoamylase at 40-65° C. for 5-60 minutes.

9. A method for obtaining a hydrolysed oat material which comprises (a) obtaining a slurry of a heat-treated oat material in water, and (b) liquefying and saccharifying the slurry of step (a) in one step at a temperature of 70-90° C. with at least one bacterial endo-alpha-amylase and at least one bacterial maltogenic alpha- amylase.

10. The method of claim 9, wherein the bacterial endo-alpha-amylase is obtained from Bacillus.

11. The method of claim 9, wherein the bacterial endo-alpha-amylase has at least 70% sequence identity to SEQ ID NO: 3.

12. The method of claim 9, wherein the bacterial maltogenic alpha-amylase is obtained from Bacillus stearothermophilus.

13. The method of claim 9, wherein the bacterial maltogenic alpha-amylase has at least 70% sequence identity to SEQ ID NO: 4.

14. The method of claim 9, wherein step (b) is performed for 5-60 minutes.

15. The method of claim 9, wherein the hydrolysed oat material obtained after step (b) comprises maltose:glucose in a ratio of at least 1 (w/w).


SEQUENCE LISTING (4 sequences)

SEQ ID NO: 1 - protein, 483 aa, Bacillus licheniformis [sequence omitted]

SEQ ID NO: 2 - protein, 489 aa, Bacillus stearothermophilus
AAPFNGTMMQYFEWYLPDDGTLWTKVANEANNLSSLGITALWLPPAYKGTSRSDVGYGVYDLYDLGEFNQKGTVRTKYGTKAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTENGFYDYLMYADLDMDHPEVVTELKNWGKWYVNTTNIDGFRLDAVKHIKFSFFPDWLSYVRSQTGKPLFTVGEYWSYDINKLHNYITKTNGTMSLFDAPLHNKFYTASKSGGAFDMRTLMTNTLMKDQPTLAVTFVDNHDTEPGQALQSWVDPWFKPLAYAFILTRQEGYPCVFYGDYYGIPQYNIPSLKSKIDPLLIARRDYAYGTQHDYLDHSDIIGWTREGVTEKPGSGLAALITDGPGGSKWMYVGKQHAGKVFYDLTGNRSDTVTINSDGWGEFKVNGGSVSVWVPRKTTVSTIA

SEQ ID NO: 3 - protein, 483 aa, Bacillus amyloliquefaciens
VNGTLMQYFEWYTPNDGQHWKRLQNDAEHLSDIGITAVWIPPAYKGLSQSDNGYGPYDLYDLGEFQQKGTVRTKYGTKSELQDAIGSLHSRNVQVYGDVVLNHKAGADATEDVTAVEVNPANRNQETSEEYQIKAWTDFRFPGRGNTYSDFKWHWYHFDGADWDESRKISRIFKFRGEGKAWDWEVSSENGNYDYLMYADVDYDHPDVVAETKKWGIWYANELSLDGFRIDAAKHIKFSFLRDWVQAVRQATGKEMFTVAEYWQNNAGKLENYLNKTSFNQSVFDVPLHFNLQAASSQGGGYDMRRLLDGTVVSRHPEKAVTFVENHDTQPGQSLESTVQTWFKPLAYAFILTRESGYPQVFYGDMYGTKGTSPKEIPSLKDNIEPILKARKEYAYGPQHDYIDHPDVIGWTREGDSSAAKSGLAALITDGPGGSKRMYAGLKNAGETWYDITGNRSDTVKIGSDGWGEFHVNDGSVSIYVQK

SEQ ID NO: 4 - protein, 686 aa, Bacillus stearothermophilus
SSSASVKGDVIYQIIIDRFYDGDTTNNNPAKSYGLYDPTKSKWKMYWGGDLEGVRQKLPYLKQLGVTTIWLSPVLDNLDTLAGTDNTGYHGYWTRDFKQIEEHFGNWTTFDTLVNDAHQNGIKVIVDFVPNHSTPFKANDSTFAEGGALYNNGTYMGNYFDDATKGYFHHNGDISNWDDRYEAQWKNFTDPAGFSLADLSQENGTIAQYLTDAAVQLVAHGADGLRIDAVKHFNSGFSKSLADKLYQKKDIFLVGEWYGDDPGTANHLEKVRYANNSGVNVLDFDLNTVIRNVFGTFTQTMYDLNNMVNQTGNEYKYKENLITFIDNHDMSRFLSVNSNKANLHQALAFILTSRGTPSIYYGTEQYMAGGNDPYNRGMMPAFDTTTTAFKEVSTLAGLRRNNAAIQYGTTTQRWINNDVYIYERKFFNDVVLVAINRNTQSSYSISGLQTALPNGSYADYLSGLLGGNGISVSNGSVASFTLAPGAVSVWQYSTSASAPQIGSVAPNMGIPGNVVTIDGKGFGTTQGTVTFGGVTATVKSWTSNRIEVYVPNMAAGLTDVKVTAGGVSSNLYSYNILSGTQTSVVFTVKSAPPTNLGDKIYLTGNIPELGNWSTDTSGAVNNAQGPLLAPNYPDWFYVFSVPAGKTIQFKFFIKRADGTIQWENGSNHVATTPTGATGNITVTWQN